(19) United States
(12) Patent Application Publication
(10) Pub. No.: US 2021/0093523 A1
(43) Pub. Date: Apr. 1, 2021

(54) ANHYDROUS MAGNETIC EYELINER COMPOSITION

(71) Applicant: L'ORÉAL, Paris (FR)

(72) Inventors: Christopher Pang, New York, NY (US); Hy Si Bui, Piscataway, NJ (US)

(73) Assignee: L'ORÉAL, Paris (FR)

(21) Appl. No.: 16/587,294

(22) Filed: Sep. 30, 2019

(51) Int. Cl. A61K 8/19; A61K 8/90; A61K 8/81; A61K 8/96; A61Q 1/10; A41G 5/02; A45D 40/26

(52) U.S. Cl. CPC A61K 8/19; A61K 8/90; A61K 8/81; A45D 2200/1072; A61Q 1/10; A41G 5/02; A45D 40/26; A61K 8/965

(57) ABSTRACT

An anhydrous magnetic eyeliner composition is disclosed, as well as a device and method using the composition. The composition includes one or more triblock copolymers, one or more hydrocarbon resins, and iron oxide. In these compositions, the triblock copolymers are present in a total amount of between 1 and 4% by weight of the composition, the hydrocarbon resins are present in an amount of between 6 and 16% by weight of the composition, and the iron oxide is present in an amount of at least 25% by weight of the composition.

### FIELD OF THE INVENTION

The present invention relates to cosmetic compositions, and specifically to magnetic eyeliner products containing (i) a triblock copolymer, a diblock copolymer, or a combination thereof; (ii) a hydrocarbon resin; and (iii) a high level of iron oxide in an anhydrous system.

### BACKGROUND

Magnetic eyeliners can be used for magnetically securing magnetic eyelashes, but failure of a magnetic eyeliner to last can lead to an embarrassing situation. However, these products tend to not have the necessary longevity or be uncomfortable to wear. As such, a magnetic eyeliner that improves the longevity and is comfortable to wear is desirable.

### BRIEF SUMMARY

A first aspect of the present disclosure is an anhydrous magnetic eyeliner composition that includes one or more triblock copolymers, diblock copolymers, or a combination thereof, one or more hydrocarbon resins, and iron oxide. In these compositions, the triblock and diblock copolymers are present in a total amount of between 1 and 4% by weight of the composition, the hydrocarbon resins are present in an amount of between 6 and 16% by weight of the composition, and the iron oxide is present in an amount of at least 25% by weight of the composition.

The composition may optionally include one or more clays, which may be present in an amount of between 6 and 14% by weight. The composition may optionally include one or more solvents. Optionally, the composition may utilize between 30% and 40% by weight of the iron oxide. In some embodiments, the hydrocarbon resin is a hydrogenated styrene/methylstyrene/indene copolymer.

In some embodiments, the ratio of the amount of hydrocarbon resin to triblock copolymer, diblock copolymer, or combination thereof is between 2.5:1 and 6:1. In some embodiments, the ratio of the amount of iron oxide to triblock copolymer, diblock copolymer, or combination thereof is between 7.5:1 and 20:1. In some embodiments, the ratio of the amount of iron oxide to hydrocarbon resin is between 2.5:1 and 4:1.

A second aspect of the present disclosure is a device that includes a reservoir covered and closed by a screw cap. The reservoir has one open end and contains the anhydrous magnetic eyeliner composition of the first aspect, while the screw cap has an applicator for applying the composition to an eyelid/eyelash interface.

A third aspect of the present disclosure is a method for improving the characteristics of magnetic eyelashes. The method includes applying to an eyelid/eyelash interface the anhydrous magnetic eyeliner composition of the first aspect, preferably with the device of the second aspect, and then applying an artificial eyelash comprising magnets to the eyelid/eyelash interface.

### BRIEF DESCRIPTION OF FIGURES

FIG. 1 is a graph illustrating rheological profiles of various compositions including one embodiment of a composition as disclosed herein.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the terms “between [two numbers]” is intended to include those two numbers. For example, “x is between 1 and 2” is intended to cover 1≤x≤2.

As used herein, the terms “include”, “includes” and “including” are meant to be non-limiting.

A first aspect of the present disclosure is an anhydrous magnetic eyeliner composition that includes one or more triblock copolymers, diblock copolymers, or a combination thereof, one or more hydrocarbon resins, and iron oxide.

Triblock/Diblock copolymers

The composition according to the invention comprises at least one triblock copolymer, diblock copolymer, or combination thereof, which is preferably a thermoplastic block copolymer, preferably hydrogenated, and is preferably chosen from styrene-ethylene/propylene-styrene copolymers, styrene-ethylene/butadiene-styrene copolymers, styrene-isoprene-styrene copolymers and styrene-butadiene-styrene copolymers. Triblock copolymers are especially sold under the names Kraton® G1650, Kraton® G1657, Kraton® D1101, Kraton® D1102 and Kraton® D1160 by the company Kraton Polymers. According to one embodiment of the present invention, the hydrocarbon-based block copolymer is a styrene-ethylene/butylene-styrene triblock copolymer. According to one preferred embodiment, the composition according to the invention comprises at least one diblock copolymer, which is preferably hydrogenated, preferably chosen from butadiene/styrene copolymers, styrene-ethylene/propylene copolymers, styrene-ethylene/butadiene copolymers and styrene-ethylene/butylene copolymers. The diblock polymers are especially sold under the Kraton® brand name by the company Kraton Polymers.

According to another aspect of the present invention, the composition contains both a triblock and a diblock copolymer. In these compositions, the active amount of triblock copolymers, diblock copolymers, or combination thereof is present in a total amount of between 1 and 4% by weight of the composition.

Hydrocarbon Resins

The hydrocarbon-based resins may be chosen from olefinic polymers of low molecular mass, which may be classified, according to the type of monomer they comprise, as indene polymers, pentadiene resins, cyclopentadiene dimer resins and terpenic resins.

The indene polymers may be chosen from polymers derived from the polymerization in major proportion of indene monomer and in minor proportion of monomers chosen from styrene, methylindene and methylstyrene, and mixtures thereof. These polymers may optionally be hydrogenated, and may have a molecular weight ranging from 200 to 1,500 g/mol.

According to at least one embodiment, the indene hydrocarbon-based polymer is a block copolymer obtained from indene and from styrene or a styrene derivative.

According to at least one embodiment, the resin is chosen from indene resins, such as the hydrogenated styrene/methylstyrene/indene copolymers sold under the name “Regalite” by the company Eastman Chemical, such as REGALITE R 1100, REGALITE R 1090, REGALITE R-7100, REGALITE R 1010 HYDROCARBON RESIN and REGALITE R 1125 HYDROCARBON RESIN, as well as those sold under the references Escorez 7105 by Exxon Chem., Nevchem 100 and Nevex 100 by Neville Chem., Norsolene S105 by Sartomer, Picco 6100 by Hercules and Resinall by Resinall Corp.

The one or more hydrocarbon resins are present in a total amount of between 6 and 16% by weight of the composition.

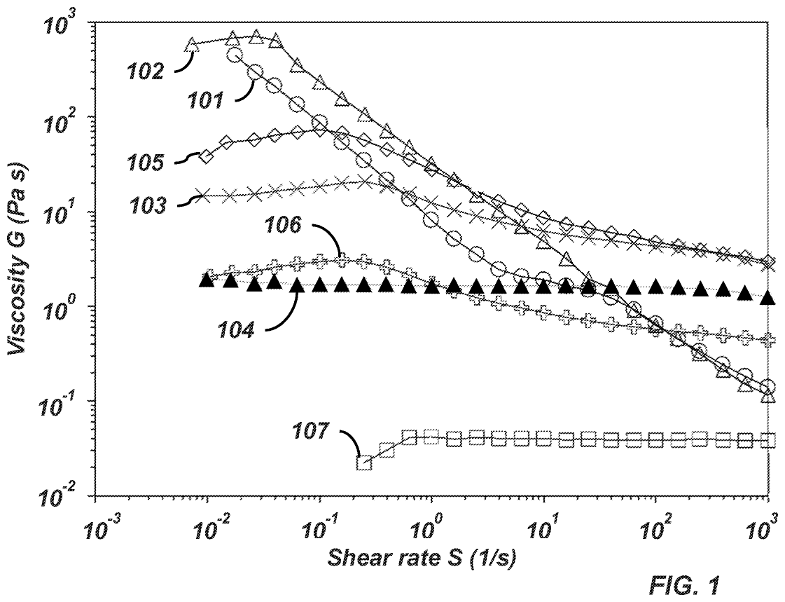

Surprisingly, the ratio of the amount of hydrocarbon resins to triblock copolymers greatly impacts the rheological profile of the composition, and the ratio can be controlled to provide a particularly desirable rheological profile. The flow curves seen in FIG. 1 were measured using a MCR 502 from ANTON PAAR rotational rheometer, equipped with an air-cooled Peltier plate to regulate the temperature. A cone and plate geometry 50 mm/angle 1° (sandblasted stainless steel at 5 μη) was used with an anti-evaporation device to avoid evaporation during measurements. The flow curves were created by stepping the shear rate from 0.01 to 1000 s-1 at 25° C. The seven variations tested include a “target” composition (101), 2% triblock copolymer/10% hydrocarbon resin (102), 6.6% triblock copolymer/10% hydrocarbon resin (103), 10% triblock copolymer/10% hydrocarbon resin (104), 6.6% triblock copolymer/10% hydrocarbon resin/5% clay (105), 5% triblock copolymer/7.4% hydrocarbon resin (106), and 5% triblock copolymer/15% hydrocarbon resin (107).

As can be seen, the profile changes dramatically when the triblock copolymer is in the 1-4 w/w % range and hydrocarbon resins are in the 6-16 w/w % range, and specifically when the ratio of hydrocarbon resin to triblock copolymer is between 2.5:1 and 6:1. At those ranges, the composition is shear thinning across the entire tested range, with a viscosity at a shear rate of 0.01 s-1 is above 100, and the viscosity at a shear rate of 500 s-1 is less than 1.

Iron Oxide

The composition also contains a colorant that has magnetic properties, which may be a magnetic or paramagnetic material. Particularly suitable pigments are nacres comprising ironoxide Fe₃O₄.

The term “nacre” means colored particles of any form, which may optionally be iridescent. More particularly, the nacres may have a yellow, pink, red, bronze, orangey, brown, gold, and/or coppery color or glint. By way of example, pigments presenting magnetic properties are those sold under the trade names COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) by MERCK, or indeed FLAMENCO TWILIGHT RED, FLAMENCO TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424 CHROMA-LITE, BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) by ENGELHARD.

Black iron oxide particles, e.g. those sold under the trade name SICOVIT noir E172 by BASF, or soft-iron based particles proposed under the trade name STAPA® WM IRON VP 041040 by ECKART, may also be mentioned.

Magnetic pigments may also comprise metallic iron, in particular passivated soft iron, e.g. obtained from carbonyl iron by implementing the method described in U.S. Pat. No. 6,589,331, the contents of which are incorporated herein by reference. The particles may include a surface oxide layer.

The iron oxide may be coated or uncoated iron oxide.

The iron oxide is present in an amount of at least 25% by weight of the composition. In certain embodiments, the composition includes between 30% and 40% by weight of the iron oxide. In preferred embodiments, the composition includes up to 50% by weight of the iron oxide. In certain embodiments, the composition includes between 25% and 50% by weight, between 30% and 50% by weight, between 35% and 50% by weight, and/or between 40% and 50% by weight of the iron oxide. In some embodiments, the composition includes up to 75% by weight of the iron oxide.

In some embodiments, the ratio of the amount of iron oxide to triblock copolymer is between 7.5:1 and 20:1. In some embodiments, the ratio of the amount of iron oxide to hydrocarbon resin is between 2.5:1 and 4:1.

Other Ingredients

The composition may optionally include other ingredients, including other colorants, clays, butters, and solvents.

Colorants

Colorants, such as organic and inorganic pigments, may be utilized. Examples of inorganic pigments include, but are not limited to, talc, mica, magnesium carbonate, calcium carbonate, magnesium silicate, aluminum magnesium silicate, silica, titanium dioxide, zinc oxide, ultramarine, polyethylene powder, methacrylate powder, polystyrene powder, silk powder, crystalline cellulose, starch, titanated mica, iron oxide titanated mica, bismuth oxychloride, and the like. Examples of organic pigments include, but are not limited to, carbon black, and barium, strontium, calcium and aluminum lakes. The aforementioned pigments can be used independently or in combination.

Clays

Clays are silicates containing a cation that may be chosen from calcium, magnesium, aluminium, sodium, potassium and lithium cations, and mixtures thereof, and the clays may be optionally modified in various fashions.

Examples of such products that may be mentioned include clays of the smectite family, and also of the vermiculite, stevensite and chlorite families. These clays can be of natural or synthetic origin.

Mention may more particularly be made of smectites, such as saponites, hectorites, montmorillonites, bentonites or beidellite and in particular synthetic hectorites (also known as laponites), such as the products sold by Laporte under the names Laponite XLG, Laponite RD and Laponite RDS (these products are sodium magnesium silicates and in particular sodium lithium magnesium silicates); bentonites, such as the product sold under the name Bentone HC by Rheox; magnesium aluminium silicates, which are in particular hydrated, such as the products sold by Vanderbilt Company under the name Veegum Ultra, Veegum HS or Veegum DGT, or also calcium silicates and in particular that in synthetic form sold by the company under the name Micro-Cel C.

Preferably, use is made of organophilic clays, more particularly modified clays, such as montmorillonite, bentonite, hectorite, attapulgite and sepiolite, and mixtures thereof. The clay is preferably a bentonite or a hectorite.

These clays are modified with a chemical compound chosen from quaternary amines, tertiary amines, amine acetates, imidazolines, amine soaps, fatty sulfates, alkylarylsulfonates and amine oxides, and mixtures thereof.

Mention may thus be made of hectorites modified with a quaternary amine, more specifically with a C₁₀ to C₂₂ fatty acid ammonium halide, such as a chloride, such as hectorite modified with distearyldimethylammonium chloride (CTFA name: Disteardimonium hectorite), for instance the product sold under the name Bentone 38V®, Bentone 38V CG or Bentone EW CE by the company Elementis, or stearalkonium hectorites, such as Bentone 27 V.

Mention may also be made of quaternium-18 bentonites, such as those sold under the names Bentone 34 by the company Elementis, Tixogel VP by the company United Catalyst and Claytone 40 by the company Southern Clay; stearalkonium bentonites, such as those sold under the names Tixogel LG by the company United Catalyst and Claytone AF and Claytone APA by the company Southern Clay; or quaternium-18/benzalkonium bentonites, such as that sold under the name Claytone HT by the company Southern Clay.

According to a preferred embodiment, the thickener is chosen from organophilic modified clays, in particular organophilic modified hectorites, in particular modified with benzyldimethylammonium stearate chloride or with distearyldimethylammonium chloride.

In accordance with one variant of the invention, the content of optionally modified clay ranges from 6% to 14% by weight relative to the weight of the composition.

Solvents

The composition may optionally include one or more solvents. The solvent may be a polyol. The solvent may be a cosmetic oil, including hydrocarbons or mixtures of hydrocarbons, such as isododecane, isohexadecane, isoparaffins, or mineral oil. The solvent may be an ester such as isopropyl palmitate, isopropyl myristate, isononyl isonanoate, C₁₂-C₁₅ alkyl benzoates, caprylic/capric triglycerides, ethylhexyl hydroxystearate, or pentaerythritol tetraoctanoate.

A second aspect of the present disclosure is a device capable of delivering the disclosed anhydrous magnetic eyeliner composition. The device includes a reservoir, usually a glass or molded plastic component, that has an opening at one end. The device also includes a screw cap that has an appropriate applicator for applying the eyeliner composition to, e.g., the portion of the eyelid that will eventually form an eyelid-eyelash interface. The screw cap is configured to cover the opening at the end of the reservoir. During manufacturing, the reservoir is filled with the eyeliner composition, after which the applicator portion of the screw cap is placed into the reservoir and the screw cap is then screwed on, closing the reservoir.

A third aspect of the present disclosure is a method for improving the characteristics (i.e., longevity, comfort, etc.) of magnetic eyelashes. The method includes applying to the portion of the eyelids that form the eyelid-eyelash interface the disclosed anhydrous magnetic eyeliner composition. Afterwards, a user then applies an artificial eyelash comprising a magnet, contacting the eyelid/eyelash interface.

### EXAMPLE

TABLE 1
Example Formulation.

| Material | Quantity (w/w %) |
|---|---|
| Triblock Copolymer | 1-4% |
| Hydrocarbon Resin | 6-16% |
| Iron Oxide | 25-75% |
| Solvent | 5-20% |

One method for manufacturing the above example formulation is as follows. Triblock copolymer was first dissolved in solvent at high temperature (60C-80C) while mixing with a cowles blade at 1000 rpm. Then hydrocarbon resins were mixed in, using a cowles blade at 1000 rpm, at room temperature until dissolved. Lastly, the pigments were mixed in, using a cowles blade at 2000 rpm, until uniform.

Testing

To illustrate the benefits of the formulation, five example formulations were prepared, altering only the amount of iron oxide in the formulation. The percentages of iron oxide used were 8, 18, 28, 38, and 48 percent iron oxide in the formulation. The eyeliners were applied directly onto a vertically-oriented test surface. Artificial lashes were then attached to the test surface, which was dropped from 4 inches and 7 inches above a table. How well each of the lashes stayed in place was then subjectively assessed (“Poor” vs. “Good” performance). The results are summarized in Table 2, below.

TABLE 2

| | Percentage Iron Oxide In Formulation | | | | |
|---|---|---|---|---|---|
| | 8 w/w % | 18 w/w % | 28 w/w % | 38 w/w % | 48 w/w % |
| 4 Inch Drop | Poor | Poor | Good | Good | Good |
| 7 Inch Drop | Poor | Poor | Poor | Good | Good |

Those skilled in the art will recognize or be able to ascertain using no more than routine experimentation, many equivalents to the specific embodiments of the invention described herein. Such equivalents are intended to be encompassed by the following claims.

1. An anhydrous magnetic eyeliner composition, comprising:
at least one triblock copolymer, diblock copolymer, or combination thereof;
at least one hydrocarbon resin; and
iron oxide,
wherein the at least one triblock copolymer, diblock copolymer, or combination thereof is present in an amount of between 1 and 4% by weight of the composition,
wherein the at least one hydrocarbon resin is present in an amount of between 6 and 16% by weight of the composition, and
wherein the iron oxide is present in an amount of at least 25% by weight of the composition.

2. The anhydrous magnetic eyeliner composition according to claim 1, further comprising at least one clay.

3. The anhydrous magnetic eyeliner composition according to claim 1, wherein the eyeliner composition includes between 6 and 14% by weight of the at least one clay.

4. The anhydrous magnetic eyeliner composition according to claim 1, wherein the eyeliner composition utilizes between 30% and 40% by weight of the iron oxide.

5. The anhydrous magnetic eyeliner composition according to claim 1, wherein the at least one hydrocarbon resin is a hydrogenated indene/methylstyrene/styrene copolymer.

6. The anhydrous magnetic eyeliner composition according to claim 1, wherein a ratio of the amount of the at least one hydrocarbon resin to the amount of the at least one triblock copolymer, diblock copolymer, or combination thereof is between 2.5:1 and 6:1.

7. The anhydrous magnetic eyeliner composition according to claim 1, wherein a ratio of the amount of the iron oxide to the amount of the at least one triblock copolymer, diblock copolymer, or combination thereof is between 7.5:1 and 20:1.

8. The anhydrous magnetic eyeliner composition according to claim 1, wherein the ratio of the amount of the iron oxide to the amount of the at least one hydrocarbon resin is between 2.5:1 and 4:1.

9. The anhydrous magnetic eyeliner composition according to claim 1, further comprising at least one solvent.

10. The anhydrous magnetic eyeliner composition according to claim 1, wherein the viscosity at 25° C. at a shear rate of 0.01 s-1 is above 100, and the viscosity at 25° C. at a shear rate of 500 s-1 is less than 1.

11. An anhydrous magnetic eyeliner device, comprising:
a reservoir containing the anhydrous magnetic eyeliner composition according to claim 1, the reservoir having an opening at one end; and
a screw cap with an applicator for applying the anhydrous magnetic eyeliner composition to an eyelid/eyelash interface, the screw cap being configured to cover the opening and close the reservoir.

12. A method for improving the characteristics of magnetic eyelashes, comprising:
applying to an eyelid/eyelash interface an anhydrous magnetic eyeliner composition according to claim 1; and
applying an artificial eyelash comprising a magnet to the eyelid/eyelash interface.
